(19) United States
(12) Patent Application Publication
(10) Pub. No.: US 2025/0297414 A1
(43) Pub. Date: Sep. 25, 2025

(54) POROUS MEMBRANES INCLUDING ELECTROSPUN FIBERS

(71) Applicant: University of Utah Research Foundation, Salt Lake City, UT (US)

(72) Inventors: Jiyoung CHANG, Salt Lake City, UT (US); Jungkyu KIM, Salt Lake City, UT (US); Noori NA, Salt Lake City, UT (US); Minju KIM, Salt Lake City, UT (US)

(21) Appl. No.: 19/085,175

(22) Filed: Mar. 20, 2025

Related U.S. Application Data
(60) Provisional application No. 63/568,119, filed on Mar. 21, 2024.

(51) Int. Cl. D04H 1/728; C12N 5/00; D01D 5/00; D04H 1/435; D04H 1/4374; D04H 1/559; D04H 1/74

(52) U.S. Cl. CPC D04H 1/728; C12N 5/0068; D01D 5/0038; D04H 1/435; D04H 1/4374; D04H 1/559; D04H 1/74; C12N 2533/40; D10B 2331/041; D10B 2401/10; D10B 2509/00

(57) ABSTRACT

A porous membrane can include a first plurality of near-field electrospun fibers that are substantially parallel one to another. A second plurality of near-field electrospun fibers can be deposited over the first plurality of fibers. The second plurality of fibers can also be substantially parallel one to another. The second plurality of fibers can be transverse to the first plurality of fibers, such that the second plurality of fibers cross the first plurality of fibers to form pores between adjacent fibers of the first plurality of fibers and adjacent fibers of the second plurality of fibers.

### CROSS REFERENCE TO RELATED APPLICATIONS

This application claims priority to U.S. Provisional Patent Application No. 63/568,119, filed Mar. 21, 2024, which is incorporated herein by reference.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### INCORPORATION BY REFERENCE STATEMENT

Not applicable.

### BACKGROUND

Polymer porous membranes find widespread applications across various fields, including water filtration, biological and biomedical applications such as tissue engineering, and drug delivery due to their flexibility, biocompatibility, cost-effectiveness, and adjustable mechanical properties. There are numerous technologies to manufacture polymeric porous membranes. Electrospinning is one of the leading technologies to manufacture these membranes due at least in part to advantages of scalability, simplicity, and huge surface-to-volume ratios. Electrospinning is a technique that utilizes an electric field to draw out fine fibers from a polymer solution or polymer melt. This can be accomplished by applying a high voltage between a nozzle containing the polymer solution or polymer melt and a grounded collector. The electric field causes the polymer to be ejected to form a jet that stretches from the nozzle to the collector. The polymer jet can become thinner as it stretches, thus forming fine fibers.

### SUMMARY

Porous membranes and methods of making porous membranes are described herein. In one example, a porous membrane can include a first plurality of near-field electrospun fibers that are substantially parallel one to another. A second plurality of near-field electrospun fibers that are substantially parallel one to another can be deposited over the first plurality of fibers. The second plurality of fibers can be transverse to the first plurality of fibers. The second plurality of fibers can cross the first plurality of fibers to form pores between adjacent fibers of the first plurality of fibers and adjacent fibers of the second plurality of fibers.

An example method of making a porous membrane can include electrospinning a first plurality of fibers using near-field electrospinning. The first plurality of fibers can be substantially parallel. The method can also include electrospinning a second plurality of fibers over the first plurality of fibers using near-field electrospinning. The second plurality of fibers can be substantially parallel to each other, and the second plurality of fibers can be transverse to the first plurality of fibers, so that the second plurality of fibers cross the first plurality of fibers to form pores between adjacent fibers of the first plurality of fibers and adjacent fibers of the second plurality of fibers.

The porous membranes can be used in various applications, including tissue chip or organ-on-a-chip applications. In one example, a tissue chip can include a substrate, a porous membrane supported by the substrate, and cultured cells supported by the porous membrane. The porous membrane can include a first plurality of near-field electrospun fibers that are substantially parallel one to another. A second plurality of near-field electrospun fibers that are substantially parallel one to another can be deposited over the first plurality of fibers. The second plurality of fibers can be transverse to the first plurality of fibers. The second plurality of fibers can cross the first plurality of fibers to form pores between adjacent fibers of the first plurality of fibers and adjacent fibers of the second plurality of fibers.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 is a face-on view of an example porous membrane in accordance with the present technology.

FIG. 2 is a face-on view of another example porous membrane in accordance with the present technology.

FIG. 3 is a face-on view of another example porous membrane in accordance with the present technology.

FIG. 4 is a face-on view of another example porous membrane in accordance with the present technology.

FIG. 5 is a face-on view of another example porous membrane in accordance with the present technology.

FIG. 6 is a face-on view of two layers to be fused together to form an example porous membrane in accordance with the present technology.

FIG. 7 is a schematic view of an example near-field electrospinning system for making porous membranes in accordance with the present technology.

FIG. 8 shows an exploded view of an example tissue chip in accordance with the present technology.

FIG. 9 shows a cross-sectional view of an example tissue chip in accordance with the present technology.

FIGS. 10A-10C show graphs of fiber diameter vs. collector speed, applied voltage, and tip to collector distance (TTCD).

FIGS. 11A-11C show SEM images of fibers deposited by near-field electrospinning and treated by a heat treatment.

FIGS. 12A-12B show graphs of normalized pore area vs. heating time at various temperatures and fiber spacing.

FIGS. 13A-13B show scanning electron microscopy (SEM) images of a four-layer membrane before and after heat treatment.

FIGS. 14A-14B show SEM images of a six-layer membrane before and after heat treatment.

FIG. 15 shows a graph of pore area frequency for a two-layer membrane, four-layer membrane, and six-layer membrane.

FIGS. 16A-16B show cell tracker images of cells cultured in an example tissue chip after 6 days and after 14 days.

FIG. 17 shows a graph of transepithelial electrical resistance (TEER) measurements after 3 days, 6 days, and 14 days.

FIG. 18 shows a graph of TEER measurements of cells cultured at 33° C. and 37° C.

FIG. 19 shows a graph of TEER measurements after 7 days, 10 days, 12, days, and 14 days.

FIG. 20 shows a graph of TEER measurements of control cells compared with cells damaged by DMSO.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a fiber” includes reference to one or more of such elements and reference to “the pore” refers to one or more of such components, while reference to “depositing” refers to one or more of such steps.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” and “at least one of A, B, or C” explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Example Embodiments

Uniform pore size in membranes can be useful in several applications. For example, uniform pore sizes ensure consistent and predictable filtration performance in filtration applications. Irregular pore sizes can lead to uneven flow rates and reduced filtration efficiency, impacting the membrane's ability to selectively separate particles based on size. Also, uniform pore sizes play a role in tissue engineering, where scaffolds are used to support cell growth and tissue regeneration. It provides a suitable microenvironment for cells, promoting uniform cell distribution, nutrient diffusion, and tissue formation. In drug delivery systems, especially in controlled-release formulations, uniform pore sizes can allow more precise control over the release rate of therapeutic agents. This uniformity ensures a more predictable and controlled drug delivery profile, contributing to the therapeutic efficacy of the system. For every application, uniform pore sizes enable the prediction and control of the membrane performance. This predictability is essential for designing and optimizing membrane systems for specific applications.

Far-field electrospinning techniques commonly result in a bulk deposition of fibers. Far-field electrospinning utilizes an ejection tip for ejecting a jet of polymer solution with a long tip-to-collector distance, which results in a whipping motion of the polymer fibers formed. The inherent randomness in deposition during this process results in non-uniform pore sizes in the membranes. This can make it difficult to form membranes with small uniform pore sizes. Smaller pores can be formed by using extended deposition times that, in turn, contribute to increased membrane thickness. However, this presents challenges, particularly in biological and biomedical applications, where individual fiber tracking and monitoring can be useful. The bulk deposition obscures the distinct characteristics of each fiber, hindering detailed analyses of diameter, orientation, and surface properties. Moreover, in studying cell behavior on electrospun membranes, the inability to discern specific fiber-cell interactions limits the attribution of cellular responses to particular fiber characteristics. This lack of resolution also complicates efforts to understand the spatial distribution of fibers within the membrane, which is useful in applications like tissue engineering.

The present technology provides membranes that can have more uniform pore sizes compared to the non-uniform pore size issues arising from random deposition. The present technology also allows for smaller pore sizes without a long-increased deposition time, which can lead to undesired membrane thickness. To accomplish this, the present technology involves using near-field electrospinning (NFES) and can also involve the controlled phase change of polymers through heating above the glass transition temperature. NFES can involve directly writing on fibers, granting precise control over the placement and alignment of individual fibers. This strategic approach induces controlled spreading of fibers, allowing for precise control over pore size, ultimately overcoming the limitations of far-field electrospinning. These membrane fabrication techniques can have good precision and efficacy in a spectrum of applications.

An example porous membrane can include a first plurality of near-field electrospun fibers that are substantially parallel one to another. A second plurality of near-field electrospun fibers that are substantially parallel one to another can be deposited over the first plurality of fibers. The second plurality of fibers can be transverse to the first plurality of fibers, such that the second plurality of fibers cross the first plurality of fibers to form pores between adjacent fibers of the first plurality of fibers and adjacent fibers of the second plurality of fibers. In some examples, the first plurality of fibers can be substantially straight and uniformly spaced one from another, and the second plurality of fibers can be substantially straight and uniformly spaced one from another. In one example, the second plurality of fibers can be orthogonal to the first plurality of fibers. In certain examples, the second plurality of fibers can be at least partially fused to the first plurality of fibers by a heat treatment.

In further examples, the first and second pluralities of fibers can have an aspect ratio of fiber width to fiber thickness from about 1.5 to about 5. Although not expressly limited, at least one of the first or second plurality of fibers can have an average fiber width from about 1 μm to about 20 μm. In one example aspect, the first plurality of fibers can have an average fiber width that is different from an average fiber width of the second plurality of fibers. In some examples, at least one of the first or second plurality of fibers have an average fiber thickness from 0.5 μm to 10 μm. In further examples, the pores have an average pore size from about 1 μm to about 100 μm. In some cases, at least 95% of the pores can have a pore size within 10% of an average pore size.

The dimensions of the fiber diameter and the sizes of the pores can be regulated through the modification of process parameters and the design of membrane patterns. The selected range of porosity for the new membrane fabrication depends on the intended application. As an example, for organ chip applications, the membrane facilitates cell-cell interaction. By employing a high porosity membrane, cell-cell interaction can be maximized or improved, enabling more effective communication and exchange between different cell types. In some cases, low porosity membranes can be beneficial when the goal is to allow cell-cell material exchange with minimal direct contact between the cells. This can be useful, for example, in studying paracrine signaling or the effects of secreted factors on neighboring cells. As another example, for bioseparation or chemical separation applications, the porosity of the membrane is directly associated with its surface-to-volume ratio. A high porosity membrane with a small pore size provides the highest surface-to-volume ratio, which increases or maximizes the surface area available for surface-material interactions. This increased surface area enhances the efficiency of separation processes, allowing for improved capture, binding, or filtration of target molecules or particles.

In certain examples, the membrane can include multiple zones having a different average pore size in each of the zones. The first plurality of fibers can be spaced at a first spacing distance, and the second plurality of fibers can be spaced at a second spacing distance that is different from the first spacing distance. The membrane can have a porosity from about 20% to about 80%.

In further examples, the membrane can also include a third plurality of near-field electrospun fibers that are substantially parallel one to another deposited over the second plurality of fibers, wherein the third plurality of fibers are transverse to the first plurality of fibers and the second plurality of fibers. In certain examples, the membrane can be a multi-layer membrane, wherein the first plurality of fibers and the second plurality of fibers form a first layer of the multi-layer membrane, and wherein the multi-layer membrane further comprises an additional layer formed by electrospinning separately from the first layer and at least partially fused to the first layer by heat treatment, wherein the additional layer comprises a third plurality of near-field electrospun fibers that are substantially parallel one to another and a fourth plurality of near-field electrospun fibers deposited over the third plurality of fibers transverse to the third plurality of fibers.

The fibers can be formed of any suitable polymer material. As non-limiting examples, at least one of the first plurality of fibers or the second plurality of fibers comprises polylactic acid (PLA), polyglycolic acid (PGA), poly(D,L-lactide-co-glycoside) (PLGA), polydimethylsiloxane (PDMS), polycarbonate (PC), polyvinylidene fluoride (PVDF), or a copolymer or combination thereof. In certain examples, at least one of the first plurality of fibers or the second plurality of fibers can include poly(D,L-lactide-co-glycoside) (PLGA). The PLGA can have a lactide to glycoside ratio from 50:50 to 90:10 and a molecular weight from about 50,000 Mw to about 120,000 Mw. In some examples, the first plurality of fibers can be made of a different polymer than the second plurality of fibers. The membrane can have an anisotropic property that is different in a direction parallel to the first plurality of fibers than in a direction parallel to the second plurality of fibers.

The present disclosure also describes methods of making a porous membrane. In one example, a method of making a porous membrane can include electrospinning a first plurality of fibers using near-field electrospinning, wherein the first plurality of fibers are substantially parallel. The method can further include electrospinning a second plurality of fibers over the first plurality of fibers using near-field electrospinning, wherein the second plurality of fibers are substantially parallel to each other, wherein the second plurality of fibers are transverse to the first plurality of fibers, such that the second plurality of fibers cross the first plurality of fibers to form pores between adjacent fibers of the first plurality of fibers and adjacent fibers of the second plurality of fibers. In some examples, the first plurality of fibers can be substantially straight and uniformly spaced one from another, and wherein the second plurality of fibers can be substantially straight and uniformly spaced one from another. In some examples, the second plurality of fibers can be orthogonal to the first plurality of fibers.

The electrospinning can include ejecting a polymer fiber from a needle tip and collecting the fiber on a moving collector. In some examples, the needle tip can be positioned at a tip to collector distance from 0.05 mm to 10 mm during the electrospinning. The collector can move at a speed from 1 mm/s to 20 mm/s during the electrospinning. In certain examples, the needle tip can have an internal diameter from 0.05 mm to 0.5 mm. Ejecting the polymer fiber can include ejecting a fluid comprising the polymer and a solvent from the needle tip, wherein at least a portion of the solvent evaporates after ejecting the fluid.

In some examples, the fluid can include the polymer at a concentration from 10 wt % to 20 wt %. In certain examples, the solvent can include 1,1,1,3,3,3-hexafluoro-2-propanol (HFIP), dimethylformamide (DMF), dimethyl sulfoxide (DMSO), or combinations thereof. Electrospinning can also include applying a voltage between the needle tip and the collector, wherein the voltage is from 500 V to 1,000 V. Additionally, a sacrificial layer can be deposited on the collector before the electrospinning, wherein the sacrificial layer comprises polyethylene oxide (PEO), or a combination thereof.

The methods can also include heat treating the porous membrane at a temperature from about 50° C. to about 100° C. The porous membrane can be heated at the temperature for a time from 5 minutes to 60 minutes. The heat treating can at least partially fuse the second plurality of fibers to the first plurality of fibers. The heat treating can increase a fiber width of the first plurality of fibers and the second plurality of fibers and can reduce a pore size of the pores. In some examples, the first and second pluralities of fibers can have an aspect ratio of fiber width to fiber thickness from about 1.5 to about 5. At least one of the first or second plurality of fibers can have an average fiber width from about 1 μm to about 20 μm. In certain examples, the first plurality of fibers can have an average fiber width that is different from an average fiber width of the second plurality of fibers. In further examples, at least one of the first or second plurality of fibers can have an average fiber thickness from 0.5 μm to 10 μm. The pores can have an average pore size from about 1 μm to about 100 μm. In some examples, at least 95% of the pores can have a pore size within 10% of an average pore size. In certain examples, the membrane can include multiple zones having a different average pore size in each of the zones. For example, average pores in a first zone can be set by varying spacing compared to an adjacent zone. The spacing can be modified by altering the distance between neighboring fibers. Layers of fibers can be strategically layered, limited to designated zones only. In some cases, the first plurality of fibers can be spaced at a first spacing distance, and the second plurality of fibers can be spaced at a second spacing distance that is different from the first spacing distance. In further examples, the membrane can have a porosity from about 20% to about 80%.

The methods can also include electrospinning a third plurality of fibers over the second plurality of fibers using near-field electrospinning, wherein the third plurality of fibers are substantially parallel one to another, wherein the third plurality of fibers are transverse to the first plurality of fibers and the second plurality of fibers. In other examples, the membrane can be a multi-layer membrane, wherein the first plurality of fibers and the second plurality of fibers form a first layer of the multi-layer membrane, and wherein the method further comprising electrospinning an additional layer separately from the first layer and at least partially fusing the additional layer to the first layer by heat treatment, wherein the additional layer comprises a third plurality of near-field electrospun fibers that are substantially parallel one to another and a fourth plurality of near-field electrospun fibers deposited over the third plurality of fibers transverse to the third plurality of fibers.

In some examples, at least one of the first plurality of fibers or the second plurality of fibers can include polylactic acid (PLA), polyglycolic acid (PGA), poly(D,L-lactide-co-glycoside) (PLGA), polydimethylsiloxane (PDMS), polycarbonate (PC), polyvinylidene fluoride (PVDF), or a copolymer or combination thereof. In certain examples, at least one of the first plurality of fibers or the second plurality of fibers can include poly(D,L-lactide-co-glycoside) (PLGA). The PLGA can have a lactide to glycoside ratio from 50:50 to 90:10 and a molecular weight from about 50,000 Mw to about 120,000 Mw. In some cases, the first plurality of fibers can be made of a different polymer than the second plurality of fibers. In further examples, the membrane can have an anisotropic property that is different in a direction parallel to the first plurality of fibers than in a direction parallel to the second plurality of fibers.

The porous membranes can be used in a variety of applications. In example application includes the construction of tissue chips, of which “organ-on-a-chip” can be one type. In one example, a tissue chip can include a substrate, a porous membrane supported by the substrate, and cultured cells supported by the porous membrane. The porous membrane can include a first plurality of near-field electrospun fibers that are substantially parallel one to another. A second plurality of near-field electrospun fibers that are substantially parallel one to another can be deposited over the first plurality of fibers. The second plurality of fibers can be transverse to the first plurality of fibers. The second plurality of fibers can cross the first plurality of fibers to form pores between adjacent fibers of the first plurality of fibers and adjacent fibers of the second plurality of fibers.

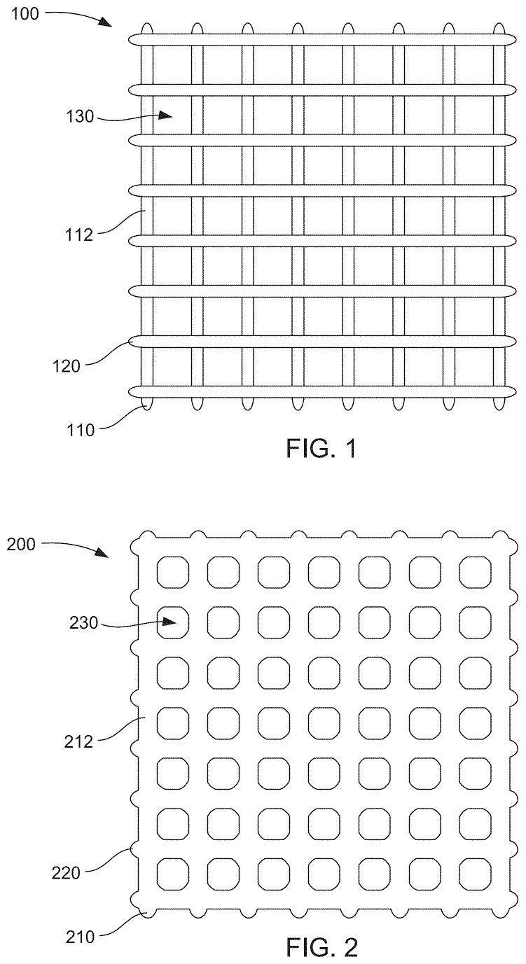

With this description in mind, FIG. 1 shows an example porous membrane 100 according to the present technology. The porous membrane includes a first plurality 110 of near-field electrospun fibers 112 that are substantially parallel one to another. In this example, the first plurality of fibers are shown in a vertical orientation. A second plurality 120 of near-field electrospun fibers are deposited over the first plurality of fibers. The second plurality of fibers are also substantially parallel on to another. However, the second plurality of fibers is oriented differently than the first plurality of fibers, so that the second plurality of fibers cross the first plurality of fibers to form pores 130 between adjacent fibers of the first plurality of fibers and adjacent fibers of the second plurality of fibers. In this example, the second plurality of fibers are shown in a horizontal orientation. In this example, the second plurality of fibers cross the first plurality of fibers to form square-shaped pores.

The near-field electrospinning process used to form the membranes described herein can allow fibers to be precisely placed. In some examples, the fibers can be formed in a substantially straight line, and many fibers can be placed substantially parallel one to another. As used herein, “substantially parallel” can allow for some imperfections. In some examples of a plurality of substantially parallel fibers, a majority of the fibers do not cross adjacent fibers. In further examples, at least 90% of the fibers do not cross adjacent fibers, or at least 95% of the fibers do not cross adjacent fibers, or at least 99% of the fibers do not cross adjacent fibers in the plurality of fibers. The substantially parallel fibers can be oriented at the same angle or at nearly the same angle as adjacent fibers. In some examples, the individual fibers can be oriented at an angle within about 10°, or within about 5°, or within about 4°, or within about 3°, or within about 2°, or within about 1° of adjacent fibers. The fibers may also be substantially straight. In some examples, an individual fiber can extend along a straight line and any segments of the fiber that are not in line with the straight line can be within about 10°, or within about 5°, or within about 4°, or within about 3°, or within about 2°, or within about 1° of the straight line. In this manner, the pore shapes can be precisely chosen. For example, pore shapes can be square, rectangular, triangular, hexagonal, octagonal, pentagonal, etc.

The fibers in the membranes described herein can be deposited much more precisely than fibers in many far-field electrospun membranes. Far-field electrospun membranes often have fibers placed in random orientations due to the uncontrolled whipping motion of the fibers during far-field electrospinning. Therefore, the far-field electrospun membranes can be a mat of randomly oriented fibers. The precise placement of fibers in the membranes described herein can allow for better control over pore size and thinner membranes. It is noted that the disclosed near-field electrospinning technique is distinct from conventional far-field electrospinning. This distinction arises primarily due to a reduction in both the distance from the tip to the collector and the voltage applied, whilst still upholding a substantial electric field. Such a configuration ensures that the material jetting from the syringe makes contact with the substrate in a direct, linear manner, as opposed to the indiscriminate deposition observed in far-field electrospinning. Accordingly, this method involves the synchronization of the stage's speed with the velocity of the ejected jet, facilitating precise material patterning. To achieve varying degrees of patterning precision, fine adjustment of both the tip-to-collector distance and the voltage can be made in accordance with the movement speed of the stage. A moveable stage with precision at the micron or sub-micron level can be used to move the collector, or to move the syringe nozzle, or a combination thereof to achieve precise control over the deposition of the fibers.

The spacing of the fibers can be adjusted to change the pore size of the membrane. Spacing fibers farther from each other can provide larger pores, and spacing fibers closer can provide smaller pores. In some examples, the spacing distance between adjacent fibers can be from about 1 μm to about 100 μm, or from about 1 μm to about 50 μm, or from about 1 μm to about 20 μm, or from about 1 μm to about 10 μm, or from about 10 μm to about 100 μm, or from about 10 μm to about 50 μm, or from about 10 μm to about 20 μm, or from about 20 μm to about 100 μm, or from about 20 μm to about 50 μm, or from about 50 μm to about 100 μm.

The width of the fibers can also affect the pore size. As used herein, the width of the fibers refers to the distance across the fiber when the membrane is viewed face-on. The thickness of the fibers refers to the dimension orthogonal to the width dimension, in the same direction as the thickness of the membrane. In some examples, the width of the fibers can be from about 1 μm to about 20 μm, or from about 1 μm to about 15 μm, or from about 1 μm to about 10 μm, or from about 1 μm to about 8 μm, or from about 1 μm to about 5 μm, or from about 5 μm to about 20 μm, or from about 5 μm to about 15 μm, or from about 5 μm to about 10 μm, from about 5 μm to about 8 μm, or from about 8 μm to about 20 μm, or from about 8 μm to about 15 μm, or from about 8 μm to about 10 μm, or from about 10 μm to about 20 μm, or from about 10 μm to about 15 μm, or from about 15 μm to about 20 μm. In certain examples, the fibers can have a uniform width. In other examples, the fibers can have varying widths, and any of the above listed widths can be average width of the fibers.

The width of the fibers can often be greater than the thickness of the fibers. Therefore, the fibers can have an aspect ratio of fiber width to fiber thickness that is greater than 1. In some examples, the aspect ratio can be from about 1.5 to about 5. When the fibers are deposited on a collector during electrospinning, the fibers may “squash” due to the electric force. This can cause the fibers to spread out in the width direction and become thinner in the thickness direction. Additionally, the fibers can spread further in the width direction if a heat treatment is applied. In further examples, the thickness of the fibers can be from 0.5 μm to 10 μm, or from 0.5 μm to 8 μm, or from 0.5 μm to 6 μm, or from 0.5 μm to 4 μm, or from 0.5 μm to 2 μm, or from 1 μm to 10 μm, or from 1 μm to 8 μm, or from 1 μm to 6 μm, or from 1 μm to 4 μm, or from 1 μm to 2 μm, or from 2 μm to 10 μm, or from 2 μm to 8 μm, or from 2 μm to 6 μm, or from 2 μm to 4 μm, or from 4 μm to 10 μm, or from 4 μm to 8 μm, or from 4 μm to 6 μm, or from 6 μm to 10 μm, or from 6 μm to 8 μm, or from 8 μm to 10 μm.

Heat treatment can be used to at least partially fuse the fibers in the membrane. As explained above, the membrane can include a first plurality of substantially parallel fibers that extend in one direction, and a second plurality of substantially parallel fibers that extend in another direction so that they cross the first plurality of fibers. These fibers can be deposited by electrospinning. After forming a membrane in this way, the membrane can be subjected to a heat treatment to at least partially fuse the fibers at their intersection points. This can strengthen the membrane, reduce the thickness of the membrane, and also reduce the pore size of the membrane. The heat treatment can cause the fibers to spread in their width direction, which in turn can make the pores smaller.

FIG. 2 shows a face-on view of another example membrane 200 that has been heat treated. The membrane includes a first plurality 210 of near-field electrospun fibers 212 that are substantially parallel one to another and a second plurality 220 of fibers deposited over the first plurality of fibers. The fibers have been subjected to a heat treatment that has caused fibers to fuse together at their intersection points. The heat treatment also causes the fibers to spread and become wider. This also causes the pores 230 to have a more rounded shape. The corners of the pores can become rounded due to surface tension/capillary forces that cause the softened polymer of the fibers to flow and form a rounded corner. Thus, the heat treatment can allow adjustment of both the shape and size of the pores.

In some examples, the heat treatment can include heating the membrane to a temperature from about 50° C. to about 100° C., or from about 50° C. to about 90° C., or from about 50° C. to about 70° C., or from about 60° C. to about 80° C., or from 70° C. to about 90° C., or from 80° C. to about 100° C. The membrane can be held at this temperature for a heating time from 5 minutes to 60 minutes, or from 5 minutes to 45 minutes, or from 5 minutes to 30 minutes, or from 5 minutes to 15 minutes, or from 15 minutes to 60 minutes, or from 15 minutes to 45 minutes, or from about 15 minutes to 30 minutes, or from 30 minutes to 60 minutes, or from 30 minutes to 45 minutes, or from 45 minutes to 60 minutes.

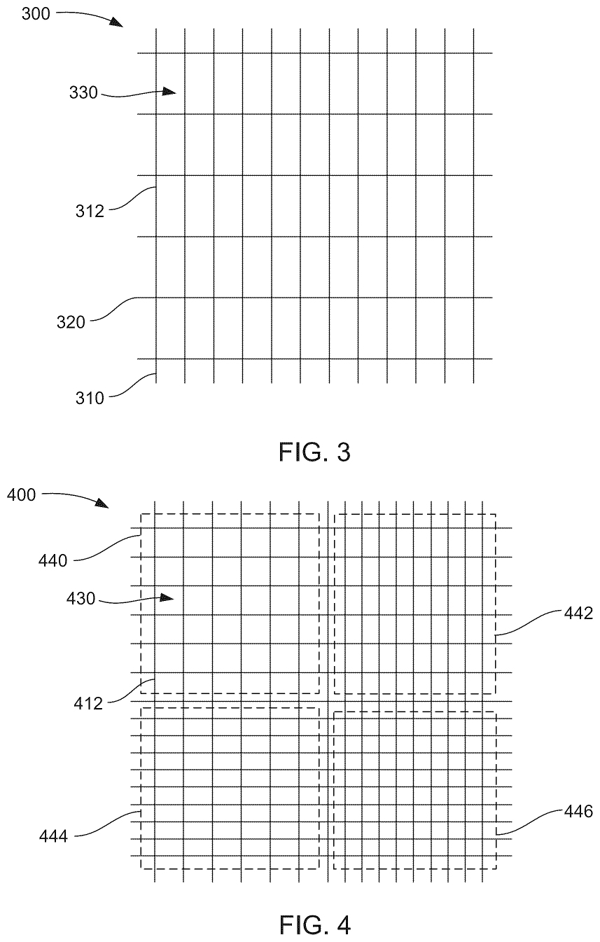

The first plurality of fibers and the second plurality of fibers can be spaced apart with uniform spacing distances in some examples. This can provide pores that have a uniform shape and size. In other examples, the first plurality of fibers can be spaced at a first uniform spacing distance, and the second plurality of fibers can be spaced at a second uniform spacing distance that is different from the first. This can result in pores that are elongated in one direction. FIG. 3 shows an example membrane 300 that has a first plurality 310 of fibers 312 spaced apart at a smaller spacing distance, and a second plurality 320 of fibers spaced apart at a larger spacing distance. The pores 330 in this example are elongated. The pore size can refer to the longest dimension of the pores.

FIG. 4 shows another example membrane 400 that includes multiple zones having different average pore sizes in each zone. In a first zone 440, the pores are large square-shaped pores 430. In a second zone 442 and third zone 444, the pores are elongated rectangle-shaped pores. In a fourth zone 446, the pores are smaller square-shaped pores. The different zones are formed by spacing the near-field electrospun fibers 412 differently in the different zones.

In certain examples, the membrane can include at least a first zone and a second zone having different average pores sizes. The first zone can make up from about 5% to about 95% of the membrane area in some examples, or from about 10% to about 90%, or from about 20% to about 80%, or from about 30% to about 70%, or from about 40% to about 60% of the membrane area in further examples. A ratio of average size of the pores in the first zone to the average size of pores in the second zone can be from about 1:10 to about 10:1, or from about 1:5 to about 5:1, or from about 1:4 to about 4:1, or from about 1:3 to about 3:1, or from about 1:2 to about 2:1 in some examples.

Some non-limiting examples of applications for membranes having multiple zones having different pore sizes can include testing an optimal membrane pore size for separating specific molecules. This can be accomplished with membranes having various pore sizes. By using a single membrane with various pore sizes, the best pore size can be determined for certain molecules by observing the local concentration of species at each location on the membrane surface. In another example, a membrane with various pore sizes on its surface can provide valuable insights into the relationship between pore size and molecular interactions. This information can guide the design and optimization of membranes for specific applications, such as protein purification, virus filtration, or drug delivery systems.

The porosity of the membrane can be the percentage of the area of the membrane that is open pores out of the entire geometric area of the membrane. In some examples, the porosity can be from about 20% to about 80%, or from about 20% to about 60%, or from about 20% to about 40%, or from about 40% to about 80%, or from about 40% to about 60%, or from about 60% to about 80%.

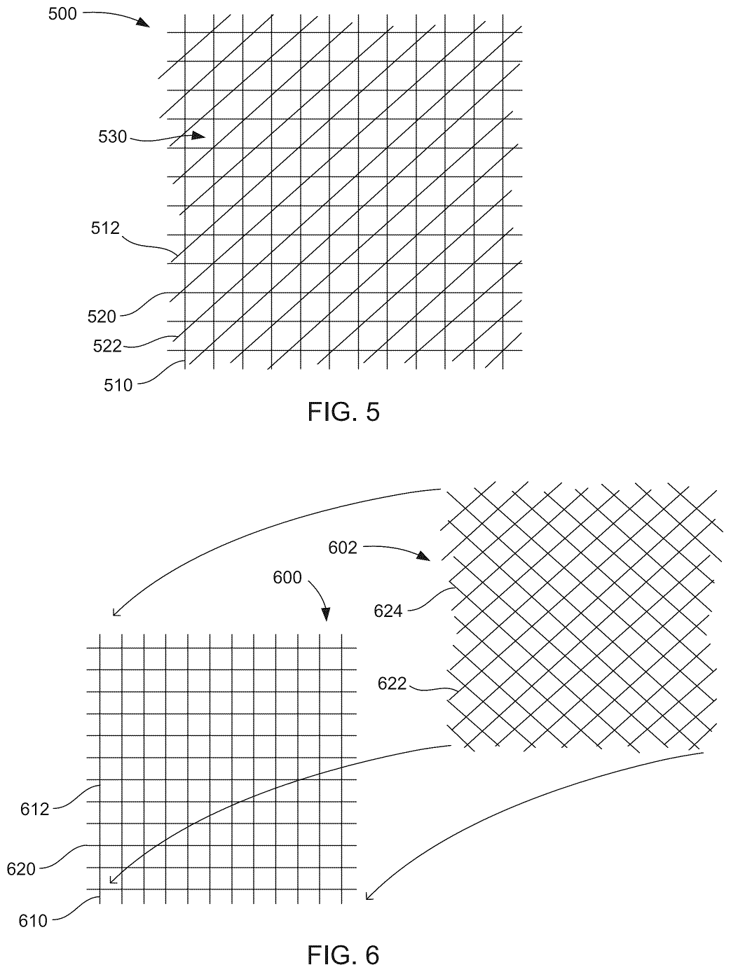

The pore size can also be adjusted by adding additional fibers to the membrane. FIG. 5 shows an example membrane 500 that includes a first plurality 510 of near-field electrospun fibers 512, which are oriented vertically, a second plurality 520 of fibers oriented horizontally, and a third plurality 522 of fibers oriented diagonally. The diagonal fibers can be oriented at any angle that is transverse to both the first plurality of fibers and the second plurality of fibers. This membrane can have a wider distribution of pore sizes of the pores 530, because the diagonal fibers form a variety of differently sized pores when crossing the first and second pluralities of fibers.

A third plurality of fibers can be deposited directly over the first and second plurality of fibers by near-field electrospinning in some examples. However, in some cases the increasing thickness of the fibers present on the collector can interfere with the electrospinning process and may reduce the precision of placing the third plurality of fibers. Fourth, fifth, and further layers of fibers can also be deposited, but the precision can be 15 affected by the thickness of the layers of fibers already on the collector.

In other examples, a multi-layer membrane can be made by forming two membranes and then fusing the two membranes together. FIG. 6 shows a particular example in which a first membrane 600 is formed from a first plurality 610 of fibers 612 and a second plurality 620 of fibers. A second membrane 602 is also formed from a third plurality 622 of fibers and a fourth plurality 624 of fibers. The second membrane can be placed overlapping the first membrane and the membranes can heat treated to at least partially fused the membranes together. Thus, a multi-layer membrane can be formed. In this example, the second membrane has diagonal fibers that will form smaller pores when the second membrane is fused to the first membrane.

The near-field electrospun fibers can be made of any polymeric material that is suitable for electrospinning. In some examples, the polymer can include polylactic acid (PLA), polyglycolic acid (PGA), poly(D,L-lactide-co-glycoside) (PLGA), polydimethylsiloxane (PDMS), polycarbonate (PC), or a copolymer or combination thereof. In certain examples, the polymer can include poly(D,L-lactide-co-glycoside) (PLGA). The PLGA can have a lactide to glycoside ratio from 50:50 to 90:10 and a molecular weight from about 50,000 Mw to about 120,000 Mw. In some examples, the first plurality of fibers can be made from a different polymer than the second plurality of fibers, while in other examples the fibers can all be made from the same polymer. In certain examples, making the first and second pluralities of fibers from different polymers can provide the membrane with an anisotropic property that is different in a direction parallel to the first plurality of fibers than in a direction parallel to the second plurality of fibers. Examples of the anisotropic property can include stiffness, elasticity, or combination thereof. In other examples, anisotropic properties can also be achieved by using different fiber thickness or fiber spacing in the first and second pluralities of fibers. Furthermore, choosing different nanofiber types can allow for customizing membrane properties such as, but not limited to, bonding with specific cells or molecules, varied stiffness, etc.

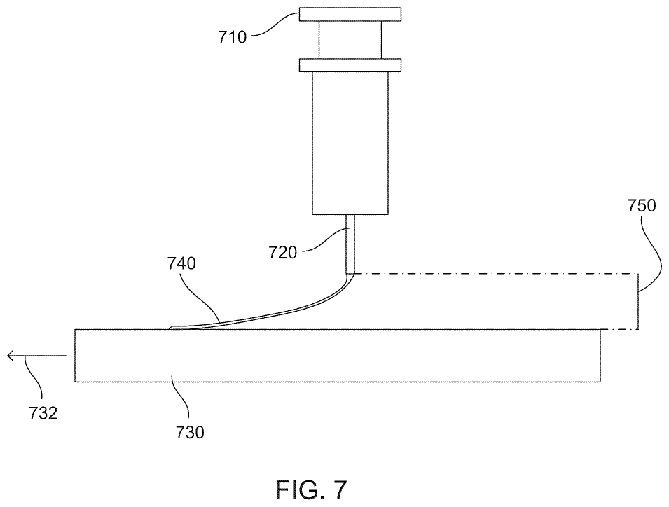

FIG. 7 shows an example near-field electrospinning system, including a syringe 710 that is filled with a polymer solution. The polymer solution can be ejected out of a needle 720 attached to the syringe. A voltage can be applied between the needle and the collector 730, which can attract the polymer fiber 740 the collector. The collector can move in the direction of the arrow 732 while the polymer is ejected. All or most of the solvent in the polymer solution can evaporate as the polymer fiber 740 travels from the needle to the collector in some examples. In other examples, a portion of the solvent can evaporate from the polymer fiber after the fiber has been deposited on the collector. By using an appropriate tip to collector distance 750 and an appropriate collector speed, the polymer fiber can be deposited in a straight line with precise control, without any random whipping motion. The collector can be moveable on two axes (only one of which is shown in this figure) to allow the polymer fibers to be deposited in a two-dimensional pattern.

In some examples, the tip to collector distance can be from 0.05 mm to 10 mm during electrospinning. In further examples, the tip to collector distance can be from 0.05 mm to 5 mm, or from 0.05 mm to 1 mm, or from 0.05 mm to 0.5 mm, or from 0.05 mm to 0.3 mm, or from 0.05 mm to 0.2 mm, or from 0.05 mm to 0.1 mm, or from 0.1 mm to 1 mm, or from 0.1 mm to 0.5 mm, or from 0.1 mm to 0.3 mm, or from 0.1 mm to 0.2 mm, or from 0.2 mm to 1 mm, or from 0.2 mm to 0.5 mm, or from 0.2 mm to 0.3 mm, or from 0.3 mm to 1 mm, or from 0.3 mm to 0.5 mm.

The collector can move on two axes to allow the fibers to be deposited in straight lines oriented in any desired direction. The movement speed of the collector can be selected so that whipping motion of the fiber is prevented, which can allow the fibers to be deposited more precisely. In some examples, the collector can move at a speed from 1 mm/s to 20 mm/s, or from 1 mm/s to 10 mm/s, or from 1 mm/s to 8 mm/s, or from 1 mm/s to 6 mm/s, or from 5 mm/s to 15 mm/s, or from 5 mm/s to 10 mm/s, or from 5 mm/s to 8 mm/s, or from 8 mms to 15 mm/s, or from 8 mm/s to 12 mm/s, or from 8 mm/s to 10 mm/s, or from 10 mm/s to 15 mm/s or from 10 mm/s to 12 mm/s.

The needle used for electrospinning can have a tip with an internal diameter from 0.05 mm to 0.5 mm in some examples. In further examples, the needle tip internal diameter can be from 0.05 mm to 0.3 mm, or from 0.05 mm to 0.2 mm, or from 0.05 mm to 0.1 mm, or from 0.1 mm to 0.5 mm, or from 0.1 mm to 0.3 mm, or from 0.1 mm to 0.2 mm, or from 0.2 mm to 0.5 mm, or from 0.2 mm to 0.3 mm, or from 0.3 mm to 0.5 mm.

The polymer used to make the fibers can be dissolved in a solvent and then the solution can be ejected from the needle tip during electrospinning. In some examples, the concentration of polymer dissolved in the solvent can be from 10 wt % to 20 wt %, or from 10 wt % to 15 wt %, or from 10 wt % to 13 wt %, or from 10 wt % to 12 wt %, or from 12 wt % to 20 wt %, or from 12 wt % to 15 wt %, or from 12 wt % to 14 wt %, or from 13 wt % to 20 wt %, or from 13 wt % to 15 wt %. In certain examples, the solvent can include 1,1,1,3,3,3-hexafluoro-2-propanol (HFIP), Dimethylformamide (DMF), Dimethyl sulfoxide (DMSO) or combinations thereof.

A voltage can be applied between the needle tip and the collector during the electrospinning process. In some examples, the voltage can be from 500 V to 1,000 V, or from 500 V to 800 V, or from 500 V to 700 V, or from 500 V to 600 V, or from 600 V to 1,000 V, or from 600 V to 800 V, or from 600 V to 700 V, or from 700 V to 1,000 V, or from 700 V to 800 V, or from 800 V to 1,000 V. In the present examples, it is observed that the manipulation of applied voltage, while maintaining a constant tip-to-collector distance, significantly influences the characteristics of the electrospinning process. Specifically, an increase in the applied voltage under these conditions results in an enhanced electric field, which subsequently increases the speed of the ejected jet and leads to the production of thinner fibers. Conversely, the application of a lower voltage, while keeping the same tip-to-collector distance, is advantageous for the precise patterning of membrane shapes and the creation of more complex patterns. This variability in voltage application allows for a versatile approach in tailoring the electrospinning process to meet specific material and design requirements.

In certain examples, a sacrificial polymer layer can be deposited on the collector before the electrospinning process. This layer can later be removed during the heat treatment. In some examples, the sacrificial layer can include polyethylene oxide (PEO), polyvinyl alcohol (PVA), or a combination thereof.

The porous membranes formed herein can be used in a variety of applications. Non-limiting examples of suitable applications can include separation membranes, biological functionalization, and anisotropic membranes. Separation membranes can be used for separating and sorting ions or molecules based on their affinity. The modification of the fiber surface in separation membrane may include, but is not limited to, the functionalization or incorporation of affinity-based materials, such as ligands, receptors, or other specific binding agents. This can enable the selective separation, purification, or concentration of targeted ions or molecules from a mixture, thereby increasing the efficiency of various applications in fields including, but not limited to, biotechnology, pharmaceuticals, environmental science, and water treatment. Furthermore, the separation membrane can be adapted to exhibit specific properties, such as hydrophilicity, hydrophobicity, charge, or size selectivity, to further enhance the separation efficiency and specificity. Additionally, the membrane can be designed for various configurations, such as flat sheet, hollow fiber, or spiral wound, and may be employed in diverse separation techniques, including, but not limited to, ultrafiltration, nanofiltration, reverse osmosis, or affinity chromatography.

Biologically functionalized membranes can be obtained by mixing with biomolecules or therapeutic reagents. A biologically functionalized membrane can comprise biomolecules or therapeutic agents for the purpose of facilitating tissue repair, wound healing, or other related applications. In addition, the innovative aspect of the present invention allows for the precise control of the release or dissolution rate of the aforementioned biomolecules or therapeutic agents, thereby optimizing their therapeutic efficacy in the targeted application.

Anisotropic membranes can use different types of polymer fibers in different directions and can be employed to create a mesh-shaped anisotropic membrane with customized strength and stiffness characteristics which vary in direction and/or location. This aspect enables customization of the mechanical properties of the anisotropic membrane, specifically in terms of strength and stiffness, by judiciously selecting and manipulating the polymer fibers and their directional arrangement within the mesh structure. The anisotropic membrane can be designed for a wide array of applications, including, but not limited to, filtration, separation, reinforcement, or load-bearing composite materials in diverse fields. In certain embodiments, the anisotropic membrane can incorporate polymer fibers with distinct physical, chemical, or mechanical properties, as well as varying fiber diameters, cross-sectional shapes, or degrees of crystallinity. The arrangement of the polymer fibers may be achieved through techniques including, but not limited to, weaving, knitting, braiding, or non-woven processes, resulting in unique anisotropic membrane designs tailored for specific applications and performance requirements.

The porous membranes can also be used in tissue chips, which can sometimes also be known as “organ-on-a-chip” or organ chips. These chips can be engineered systems that mimic some structural and functional characteristics of tissues or organs. In some examples, tissue chips can include human cells such as brain cells, heart cells, kidney cells, liver cells, skin cells, and others. The cells can be cultured on a porous membrane as described herein. These porous membranes can be particularly useful for culturing cells due to the high pore uniformity and low thickness of the membranes. The porous membrane can allow for maximized cell-to-cell interaction and permeability.

In certain examples, kidney cells can be cultured on the porous membrane. In more specific examples, kidney glomerular cells can be cultured on the porous membrane. In further examples, a combination of multiple different types of cells can be cultured on the porous membrane. In certain examples, one of the types of cells can include endothelial cells. In a specific example, kidney glomerular cells and endothelial cells can be cultured on a single porous membrane. In some examples, a first type of cells can be placed in contact with one side of the membrane, and a second type of cells can be placed in contact with the other side of the membrane.

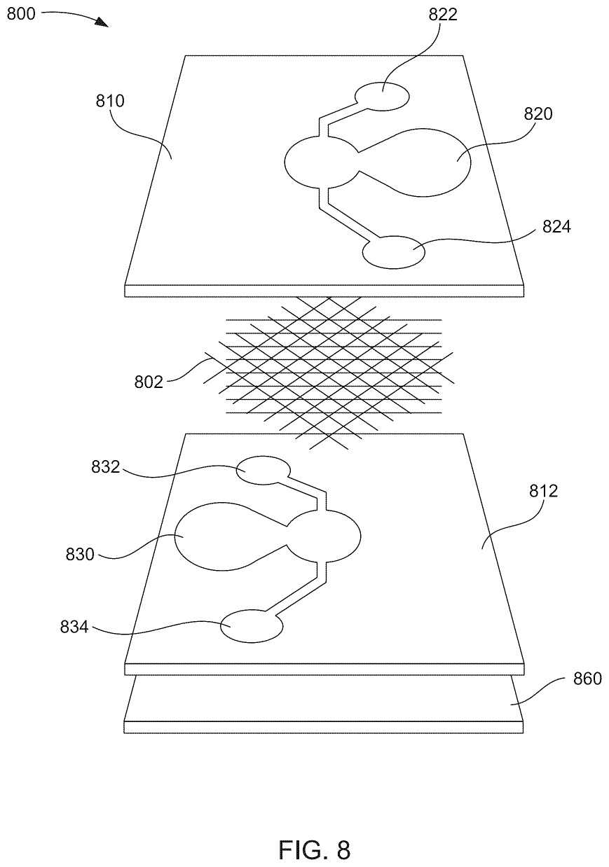

FIG. 8 shows an exploded view of an example tissue chip 800 that can be constructed using a porous membrane 802 as described herein. The tissue chip includes a substrate made up of a top microfluidic layer 810 and a bottom microfluidic layer 812. The porous membrane is held between these layers. The top microfluidic layer includes a top chamber 820 and an inlet 822 into the top chamber and an outlet 824 from the top chamber. The bottom microfluidic layer includes a bottom chamber 830 and an inlet 832 to the bottom chamber and an outlet 834 from the bottom chamber. The top chamber and bottom chamber have a central opening where the porous membrane separates the top chamber from the bottom chamber. A fluid containing cells can be introduced into one or both of the chambers and the cells can be cultured on the porous membrane. In certain examples, a first fluid containing a first type of cells can be introduced into the top chamber and a second fluid containing a second type of cells can be introduced into the bottom chamber. Both types of cells can then be cultured on the porous membrane. The tissue chip also includes a glass substrate 860 under the bottom microfluidic layer.

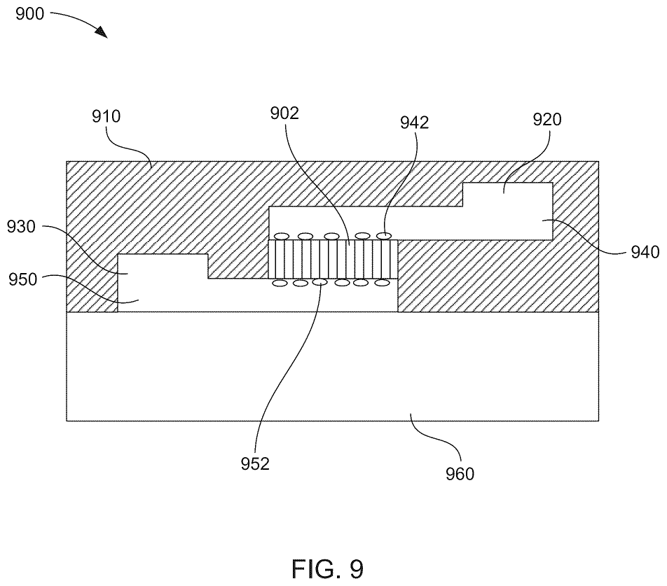

FIG. 9 shows a cross-sectional view of another example tissue chip 900 with a similar design. This example includes a microfluidic substrate 910 on a glass substrate 960. The microfluidic substrate includes a top chamber 920 and a bottom chamber 930. A porous membrane 902 separates the top chamber from the bottom chamber. The top chamber contains a first fluid 940 containing a first type of cells 942. The bottom chamber contains a second fluid 950 containing a second type of cells 952. The cells are cultured on the top and bottom surfaces of the porous membrane.

In further examples, tissue chips can include a substrate having a variety of other designs and configurations. The substrate can be a solid material that supports the porous membrane. In certain examples, the substrate can include a fluid chamber, fluid channels, or a combination thereof. The fluid chamber and fluid channels can be microfluidic features in some cases. Microfluidic channels and microfluidic chambers can have at least one dimension that is on the micron scale, such as less than 1,000 μm, or less than 100 μm, or less than 10 μm, or about 1 μm. In certain examples, the microfluidic channels or chambers can have a height or width that is on the micron scale. The microfluidic channels or chambers can be designed to place a fluid containing cells in contact with the porous membrane. In further examples, the substrate can include fluid chambers, channels, or a combination thereof configured to place two or more different fluids in contact with the porous membrane. The different fluids can contain any combination of a single type of cells, two types of cells, or more than two types of cells.

Any suitable materials and manufacturing techniques can be used to make the substrate. In some examples, microfluidic features can be formed by machining, molding, casting, photolithography, additive manufacturing, or other techniques. The substrate can comprise a solid material such as glass, plastic, acrylic, polypropylene, a photoresist such as SU8, metal, or another suitable material.

The described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

### Examples

Poly(D,L-lactide-co-glycolide) (PLGA), a synthetic copolymer derived from polylactic acid (PLA) and polyglycolic acid (PGA), has garnered significant attention as a preferred material for scaffold construction. This popularity is attributed to its favorable characteristics, including the ability to tailor degradation rates, exceptional mechanical strength and toughness, and straightforward processing. Moreover, PLGA exhibits the advantage of being easily electrospun into fiber form. In the current example, a PLGA solution with a concentration of 13 wt % was selected after a thorough comparison with other weight percentages, demonstrating superior uniformity in the solution. This concentration was deemed optimal for subsequent fabrication processes.

### Solution Preparation

Poly(D,L-lactide-co-glycolide) (PLGA 75:25) with a molecular weight ranging from 66,000 to 107,000 and 1,1,1,3,3,3-Hexafluoro-2-propanol (HFP) were procured from Sigma-Aldrich. PLGA pellets, constituting 13 wt % of the solution, were introduced into HFP and thoroughly mixed for 24 hours at room temperature.

### Nearfield Electrospinning (NFES)

PLGA membranes were fabricated using a Nearfield Electrospinning (NFES) system, comprising an XYZ controllable stage (ONE-XY100 for XY axis, one μm resolution, GTS30 V for Z-axis, 0.1 μm resolution, Newport), a long-distance microscope (K2 Distamax, Infiniti, Basler ace), a voltage supply (PS350, Standford Research System), a USB camera (Koolertron), and a pneumatic pump (Ultimus V dispenser, Nordson). These components were meticulously coordinated through a LABVIEW (National Instruments) graphical user interface (GUI) program.

A PLGA polymer solution was injected into a 3 cc syringe with a 30-gauge needle to achieve the desired fiber diameter. The top part of the syringe was connected to the pneumatic pump with an airflow to maintain a droplet on the tip. Various experimental factors were examined to assess the impact on fiber diameter, including collector speed (6, 8, 10 mm/s), applied voltage (600, 700, 800, 900, 1000V), and tip-to-collector distance (0.1, 0.2, 0.3 mm). Conditions of 600V, a collector speed of 10 mm/s, and a tip-to-collector distance of 1 mm were selected based on the uniformity and the suitable for membrane applications, which the average fiber diameter ranging from 8-10 μm.

### Fabrication of PLGA Membrane

After determining the optimal parameters for manufacturing PLGA fibers, membranes were fabricated with different spacings (50, 70, 100 μm) across the XY axis to investigate the influence of temperature on pore area.

### Observation of Pore Area

Upon manufacturing the grid-shaped membranes, pore areas were observed using a CCD camera (MU1403, AmScope) and AmScope software in conjunction with a microscope (M Plan Apo 5, Mitutoyo). The membranes were subjected to varying temperatures on a hotplate for one hour, and the temperature was validated using an infrared thermometer. Images of the same membrane area were captured every 15 minutes to monitor changes in pore areas.

### Measurement Using Scanning Electron Microscope (SEM)

To examine the cross-section of fibers after one hour of heat treatment at different temperatures, the SEM (FEI Quanta 600 FE-ESEM) technique was employed. Ten PLGA fibers were deposited on four individual silicon wafers. One sample was maintained at room temperature, while the other three samples were heated at 50° C., 70° C., and 90° C., followed by immersion in liquid nitrogen and subsequent fracturing to observe fiber cross-sections. Before measurements, the samples were coated with an approximately 10 nm layer of platinum-gold to prevent charging effects during SEM operation.

### Observation of Multilayer Membrane

In this experimental setup, two, four, six, and eight-layer configurations of membranes were superimposed at distinct angular orientations (˜45° for the four-layer configuration and ˜30° and ˜60° for the six-layer configuration, and ˜20° each for eight-layer configuration). The multilayer membrane was observed using a CCD camera (MU1403, AmScope) and AmScope software in conjunction with a microscope (M Plan Apo 5, Mitutoyo). The objective was to illustrate variations in pore shapes and sizes resulting from the specific overlapping angles. Following each layer's precise alignment and superimposition, the composite membranes were subjected to heat treatment on a hotplate for an hour. This thermal treatment aimed to induce the merging of individual layers, consolidating them into a singular, integrated layer. The determination of pore size distribution for each configuration was conducted using Image J software. A total of ten photographs were captured randomly from three distinct membrane samples for each configuration, and subsequent analysis was performed to ascertain the distribution of pore sizes.

### Optimization of NFES Parameters for PLGA Fiber Fabrication

To ensure the production of a uniformly porous membrane, the PLGA fiber manufacturing parameters can be optimized. The nanofiber electrospinning process allows for the adjustment of key parameters such as voltage, pressure, tip-to-collector distance (TTCD), and collector speed, influencing the diameter of the resulting fibers. These parameters were systematically managed through a customized LABVIEW graphical user interface (GUI).

In order to investigate the impact of these parameters on fiber diameter and, consequently, pore size, a comprehensive exploration was conducted following the parameters outlined in Table 1. The pressure was minimized to facilitate droplet formation at the needle tip, while the solution concentration remained fixed at 13 wt %, chosen for its superior solution uniformity. Fiber diameter assessment involved measuring the average diameter of 10 fibers for each parameter, excluding any irregular fibers.

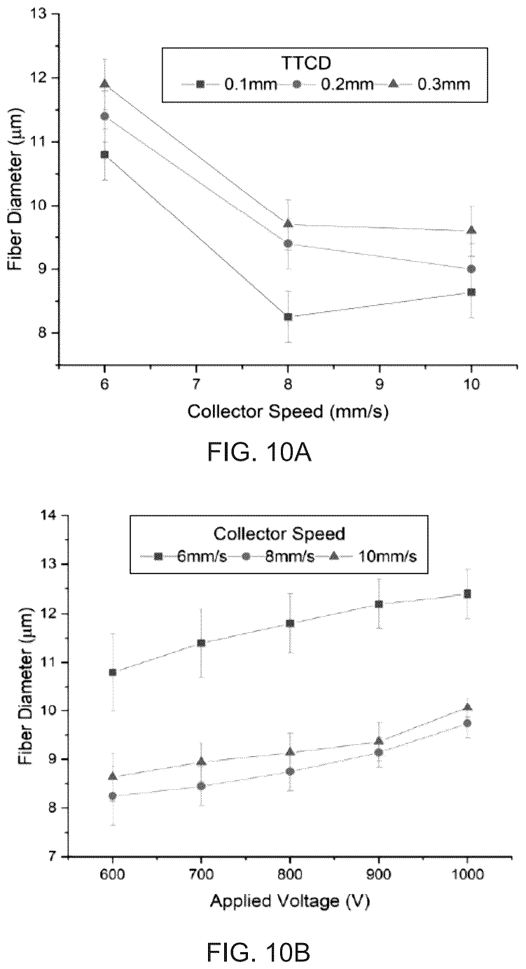

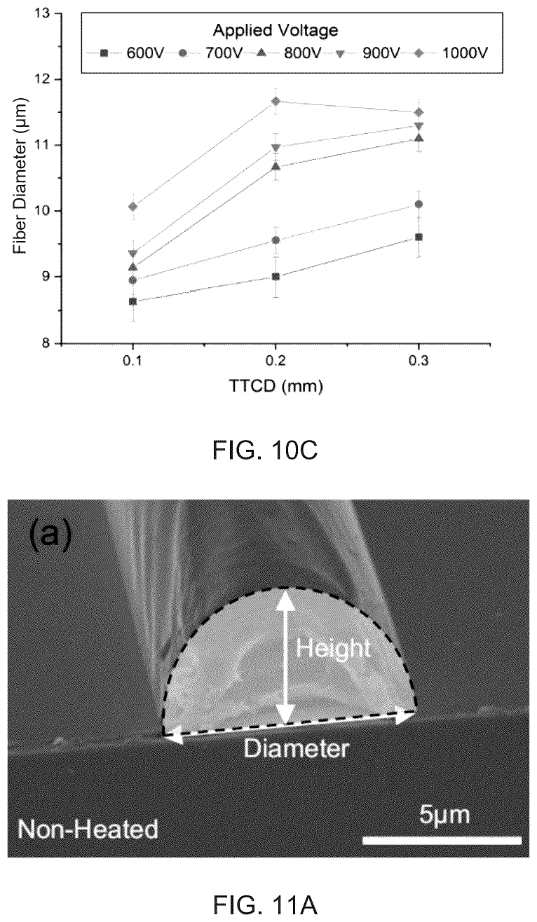

In the example NFES system, the applied voltage, TTCD, and the collector velocity can be easily adjusted using LABVIEW GUI. In this example, the applied voltage was adjustable from 600 V to 1,000 V by intervals of 100 V; the TTCD was adjustable from 0.1 mm to 0.3 mm by intervals of 0.1 mm; and the collector speed was adjustable from 6 mm/s to 10 mm/s by intervals of 2 mm/s. FIGS. 10A-10C depict the influence of individual parameters on fiber diameter, with the other parameters fixed at 600V applied voltage, 0.1 mm TTCD, and 10 mm/s collector speed. FIG. 10A indicates that an increase in collector speed results in a thinner fiber diameter, although beyond 8 mm/s, the fiber diameter exhibits minimal change for all TTCD values. FIG. 10B demonstrates that an increase in applied voltage corresponds to an increase in fiber diameter, regardless of collector speed. FIG. 10C reveals that an increase in TTCD leads to an increase in fiber diameter for all applied voltages. An analysis of variance (ANOVA) test indicated the significance of all three parameters. However, TTCD emerged as the least significant among them. Due to the uniformity observed in the fibers and the desired fiber diameter ranging from 8-10 μm, the following parameters were selected: 600 V applied voltage, 0.1 mm TTCD, and 10 mm/s collector speed. These parameters were deemed optimal for subsequent membrane fabrication processes.

Analysis of Polymeric Fiber Morphology with Temperature Variation

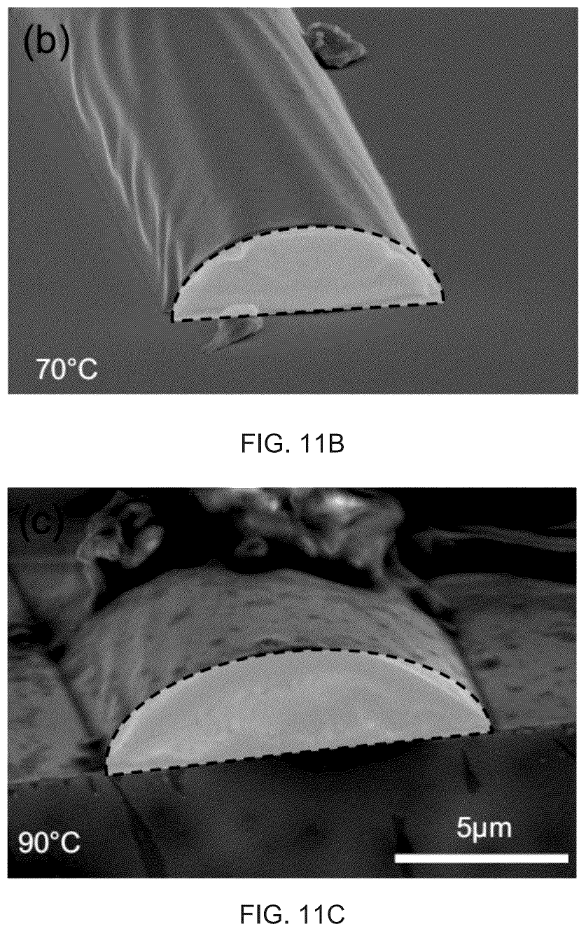

Polymeric fibers undergo a transformative process as they approach or surpass their glass transition temperature, resulting in notable modifications to their cross-sectional shape. FIGS. 11A-11C visually capture the impact of different temperatures on fiber formation. Upon subjecting fibers to a 1-hour heat treatment at diverse temperatures and subsequent scanning electron microscope (SEM) examination, the height and diameter ratio of the fibers were determined by averaging measurements from approximately ten fibers for each temperature. The non-heated fibers had an average height/diameter ratio of 0.46; the fibers heated at 50° C. had a ratio of 0.45; the fibers heated at 70° C. had a ratio of 0.37; and the fibers heated at 90° C. had a ratio of 0.25. Remarkably, a discernible correlation emerged between elevated temperatures and increased fiber diameter, concomitant with reduced fiber height. This diminishment in height is ascribed to the polymer's transition into a rubbery state, characterized by heightened molecular mobility and a relaxation of the molecular structure supporting the fiber height.

The fibers spread due to the polymer's transition into a rubbery state, facilitated by the increased molecular mobility. This enhanced flexibility allows the fibers to spread and adopt a larger cross-sectional diameter. The initial rapid spreading within the first 15 minutes is attributed to the heightened molecular mobility during this period, indicative of the transition phase. As time progresses, the gradual spreading is influenced by the limited volume of the fibers, mitigating the extent of the spreading process. These findings underscore the substantial influence of heat treatment above the glass transition temperature on the morphological characteristics of PLGA fibers. The detailed analysis presented here clarifies the dynamic interplay between temperature-induced transitions and the resulting fiber morphology.

Analysis of Pore Area Difference with Temperature Variation

Controlling pore size can be useful in membrane fabrication. As previously discussed, individual fibers exhibit a spreading behavior when exposed to temperatures exceeding the polymer's glass transition temperature. Conversely, when two layers of fibers are patterned, the intersections in the x-y plane form junctions. Application of heat to the membrane results in the merging and spreading of these junctions, thereby establishing a robust bond and reducing overall membrane thickness. This phenomenon serves as a means to govern membrane porosity.

To investigate the influence of fiber spacing on membrane characteristics, membranes with different spacings (50, 70, 100 μm) were fabricated. For experimental purposes, fibers with these spacings were subjected to temperatures of 50° C., 70° C., and 90° C. for an hour. The resulting membranes were photographed at 15-minute intervals.

At 50° C., minimal changes in pore sizes were observed across all three spacings. At 70° C., slight diameter spreading was noted, and the junctions between fibers began to fuse, creating a bond. Notably, at 90° C., significant fiber spreading occurred, with complete fusion of junctions. Smaller spacing correlates with increased changes in pore size area.

These phenomena can be elucidated by considering a combination of factors, including polymer behavior, thermal effects, and capillary action. As PLGA undergoes thermal softening above its glass transition temperature, the fibers become more flexible, facilitating their spreading. Elevated temperatures induce molecular mobility, potentially leading to partial melting and fusion of fibers at junctions. Capillary action, the ability of a liquid to flow in narrow spaces without external force, is also implicated. Smaller spaces between fibers result in higher capillary pressure, further influencing pore size. In essence, the interplay of spacing and temperature provides a mechanism for tuning membrane porosity.

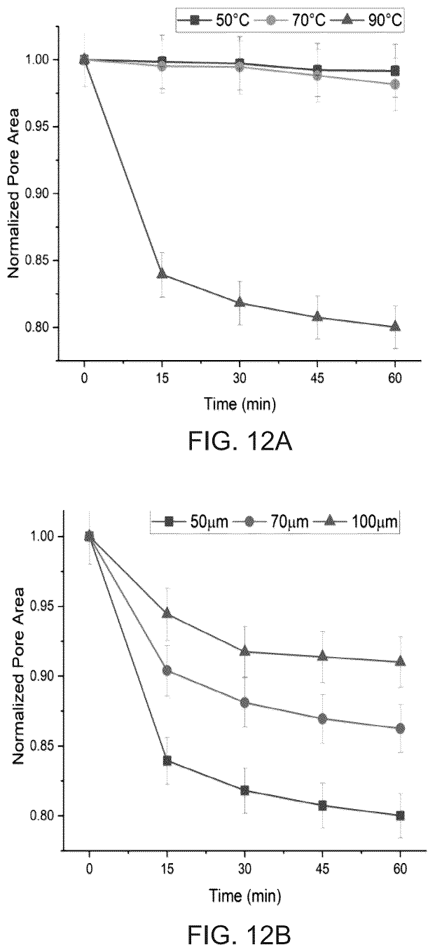

Using ImageJ software, an in-depth analysis of pore surface areas was conducted on membranes with varied spacings subjected to temperatures of 50° C., 70° C., and 90° C. FIG. 12A elucidates the temperature-dependent changes in pore area at 50 μm spacing, while FIG. 12B delineates alterations in pore area at 90° C. for different spacings. The results underscore a noticeable augmentation in pore size, particularly within the initial 15 minutes, aligning with the rapid changes observed in the spreading behavior of individual fibers under elevated temperatures. The quantitative assessment involved computing the percentage change in pore area following a 1-hour exposure. For the 50 μm spacing, the changes were 1.0%, 2.2%, and 29.3% at 50° C., 70° C., and 90° C., respectively, revealing a temperature dependent trend with higher temperatures inducing more pronounced and accelerated changes in pore size early in the exposure. The observed changes at 90° C. exhibited distinct magnitudes for different spacings, with percentages recorded as 29.3%, 15.2%, and 10.5% for spacings of 50 μm, 70 μm, and 100 μm, respectively. The variations in capillary flow at junctions contribute significantly to these trends. Smaller fiber spacings, such as 50 μm, exhibit enhanced capillary action, leading to more substantial merging and spreading at the junctions, resulting in higher percentage differences in pore area compared to larger spacings, such as 100 μm, where capillary flow is comparatively less pronounced, yielding lower percentage differences in pore area.

This non-linear relationship underscores the intricate interplay between capillary flow and fiber spacing during the thermal treatment. The capillary action at the junctions of the fibers becomes a governing factor in the redistribution of the polymer material, influencing the final morphology of the pores in a spacing-dependent manner. This insight into capillary dynamics contributes to a more comprehensive understanding of the mechanisms dictating the changes in pore area in response to thermal treatment, providing valuable guidance for optimizing membrane fabrication processes.

Manipulating the pore size in PLGA membranes involves a nuanced control of various fabrication parameters. Pore dimension reduction can be achieved through strategic adjustments, such as decreasing inter-fiber spacing or increasing individual fiber diameters. The tuning of manufacturing parameters, encompassing variables such as the poly(lactic acid) (PLA) to poly(glycolic acid) (PGA) ratio, PLGA weight percentage (wt %), and manipulating the parameters during the electrospinning process, contributes to the modulation of membrane morphology.

Specifically, a decreased spacing between adjacent fibers influences the overall porosity by restricting the available space for pore formation. Simultaneously, an increase in fiber diameter contributes to a reduction in pore size, owing to the inherently smaller inter-fiber spaces generated by larger fiber dimensions.

Furthermore, the anisotropic manipulation of membrane architecture is attainable by employing different spacings along the X and Y axes during fabrication. This deliberate spatial variation results in membranes with directional disparities in pore sizes, offering a versatile approach to tailor membrane properties based on specific application requirements.

Beyond parameter adjustments within the PLGA system, the thermal properties of alternative polymers present an avenue for tailoring pore sizes. A diversified range of pore sizes can be achieved by harnessing the distinct thermal characteristics of polymers, such as their glass transition temperatures (Tg) or melting points. This approach extends the scope of membrane design, enabling customization by selecting polymers with thermal attributes aligned with the desired membrane characteristics.

In essence, the meticulous control of manufacturing parameters and the strategic incorporation of alternative polymers with tailored thermal properties delineate a sophisticated framework for engineering PLGA membranes with finely tuned pore sizes. This multifaceted approach provides a comprehensive toolkit for addressing diverse applications in controlled-release systems, tissue engineering, and other domains where precise control over membrane porosity is paramount.

### Multilayer Membrane Infusion

The manipulation of pore size within a fibrous structure can be achieved through various methodologies, such as adjusting the inter-fiber spacing and employing larger-diameter fibers during manufacturing. Additionally, the introduction of multiple layers at different angles offers a versatile means to regulate pore size and influence the overall geometric configuration of the pores.

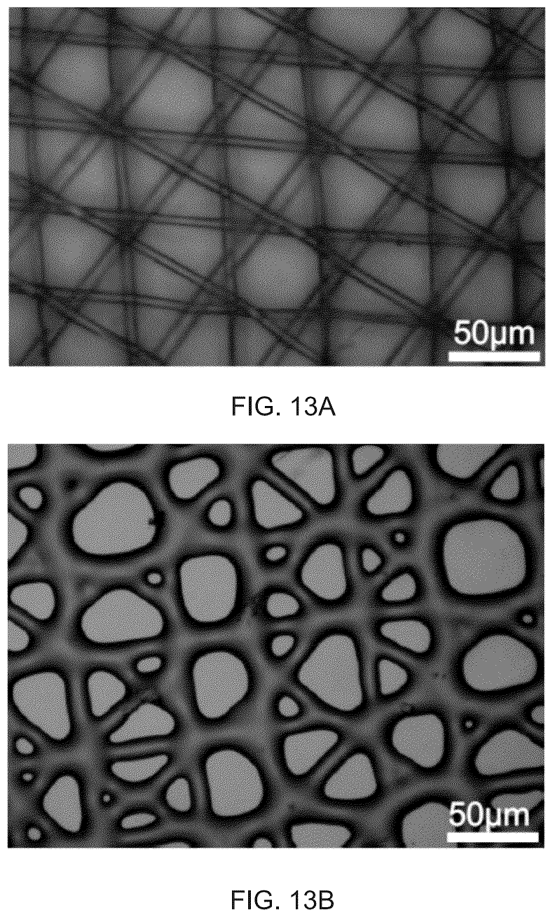

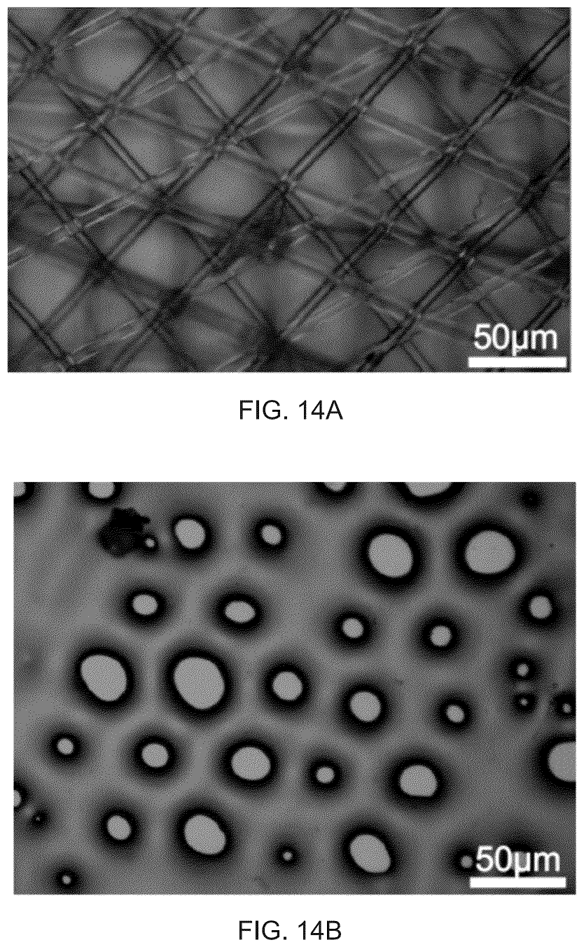

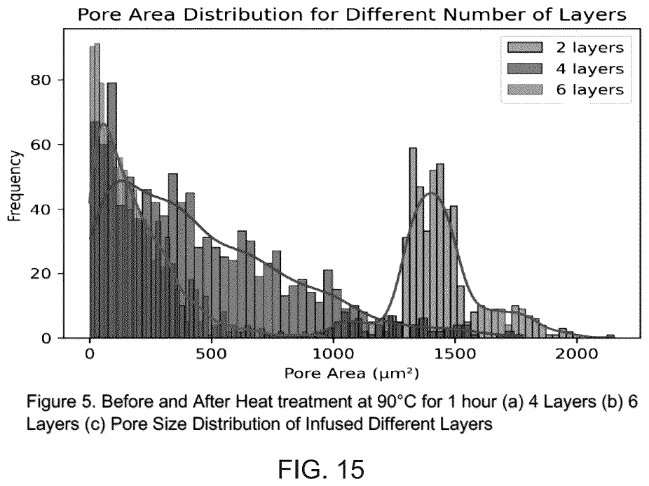

Illustratively, as depicted in FIGS. 13A-13B, the implementation of a four-layer infusion at an elevated temperature of 90° C. results in the emergence of a distinctive triangular pore morphology. FIG. 13A is a SEM image of a four-layer membrane before heat treatment, and FIG. 13B is a SEM image of the membrane after the heat treatment. In FIGS. 14A-14B, a six-layer infusion, conducted under identical temperature conditions, manifests a more compact and circular pore configuration. FIG. 14A is a SEM image of the six-layer membrane before heat treatment, and FIG. 14B is a SEM image of the membrane after the heat treatment. The delineation of pore areas across distinct infusion layers is meticulously presented in FIG. 15. Notably, the pore areas associated with more than six-layer infusions predominantly fall below 100 μm², closely resembling a circular shape with an estimated diameter of approximately 10 μm. However, an observable trend emerged as more layers were infused into the membrane, the pore area decreased, likely due to the increased density of fibers. Interestingly, as the fibers melted during the process, they contributed to filling up the pore spaces, resulting in a reduction in the overall number of pores. In contrast, the triangular morphology observed in four-layer pore areas exhibits a wider area distribution. This distinctive feature can be attributed to the infusion being executed at a 45° angle, resulting in predominantly triangular shapes. However, it is noteworthy that the imperfect infusion at this specific angle contributes to a more extensive distribution of pore areas. This phenomenon is primarily a consequence of the small holes formed during the imperfect infusion process at the 45° angle, which introduces variability in the size and shape of the resulting pores. For two layer infusions, the distribution of pore areas is centered around 1200-1400 μm², corresponding to an approximate length of 35-37 μm in the context of a square. This observation underscores the nuanced interplay between the infusion angle and inter-fiber spacing, influencing the resultant pore morphology and area distribution.

Cells were cultured on both non-heated and heated (90° C.) six-layer membranes. CellTracker™ CMFDA stained HCK cell images were captured with 20× and 40× magnification, 6 hours after initial seeding and 72 hours after initial seeding. The images showed that cell growth occurred on both the heated and non-heated membranes. The number of cells present on the membrane increased from the 6-hour image to the 72-hour image.

An alternative approach to the merger of two-layer membranes at various angles involves the direct patterning on top of each layer. In this context, the third layer was intricately patterned directly onto the second layer at a 45° angle. in the supplementary material. This method, while offering a more precise and accurate means of patterning, introduces a consideration regarding the impact of increased layering on near-field electrospinning (NFES) characteristics. As layers accumulate, the electric field undergoes changes, subsequently influencing the NFES characteristics. This highlights the intricate balance between achieving precision in patterning and acknowledging the evolving electrospinning dynamics with each additional layer, thereby involving a nuanced approach in the optimization of layering strategies for near-field electrospinning applications.

This study demonstrates the present method's efficacy in altering pore size and shape. The cumulative impact of layering and the orientation of infused fibers are shown to be factors influencing pore morphology. It is observed that an increase in the number of layers correlates with a reduction in pore size. For precise control over pore characteristics, precise programming in manufacturing can be used, dictating the dimensions and arrangement of fibers to attain the desired pore shape and size.

In conclusion, the experiments advance the understanding of the intricate interplay between fiber manufacturing parameters, temperature-induced transitions, fiber spacing, and the influence of multilayer infusion in achieving precise control over pore sizes in PLGA membranes. The optimization of PLGA fiber manufacturing parameters is identified as a fundamental prerequisite, with a focus on achieving uniform fibers to enable the subsequent creation of membranes with consistent and adjustable porosity. The NFES process, facilitated by a LABVIEW graphical user interface, allows for meticulous adjustments in critical parameters such as applied voltage, tip-to-collector distance (TTCD), collector speed, and infusion layering, with the overarching objective of influencing fiber diameter and pore shape.

NFES parameters were explored and optimal conditions for a membrane fabrication process were found to include: an applied voltage of 600 V, TTCD of 0.1 mm, and collector speed of 10 mm/s. Combined with multilayer infusion, these parameters ensured uniform fiber morphology, precisely attaining a desired fiber diameter ranging from 8 to 10 μm and influencing pore shape.

The investigation into the morphological transformations of polymeric fibers under varying temperatures provides understanding of how pore size can be controlled in the final membrane. With increasing temperature, fibers transitioned into a rubbery state characterized by heightened molecular mobility, facilitating fiber spreading and increasing cross-sectional diameter. This dynamic transition in fiber morphology and multilayer infusion was useful for achieving controlled and tunable pore sizes in subsequent membrane fabrication.

Further exploration into the impact of fiber spacing on membrane characteristics unveiled a direct correlation between smaller spacings and increased changes in pore size area. These observations were attributed to various factors, including polymer behavior, molecular mobility, and capillary action. Insights gained provide a mechanistic understanding of pore size control and serve as a basis for optimizing membrane porosity. Quantitative assessment of pore surface areas using ImageJ software confirmed temperature-dependent trends, with higher temperatures inducing more pronounced and accelerated changes in pore size, particularly within the initial 15 minutes of exposure. Additionally, the analysis demonstrated a discernible dependence on inter-fiber spacing, with smaller spacings exhibiting more substantial alterations in pore area.

Incorporating multilayer observations further enriches the understanding, demonstrating that adding layers influences not only pore size but also the overall shape of the pores. The established optimal parameters and insights contribute valuable information for future membrane fabrication processes, ensuring the production of membranes with consistent and tunable porosity for various applications.

### Controlling Biodegradability

Additionally, the electrospun PLGA membranes described herein can exhibit controlled biodegradability, which is particularly advantageous for biological and biomedical applications. The biodegradation rate of the membrane can be influenced by several factors, including the heat treatment applied during manufacturing. Notably, heat-treated fibers have been observed to show accelerated degradation profiles compared to non-heat-treated counterparts. This phenomenon can be attributed to the morphological changes that occur during the heat treatment process, where the spreading of fibers increases the surface area exposed to hydrolytic degradation while potentially reducing crystallinity within the polymer structure. The biodegradation rate can be further tailored by adjusting the lactide to glycolide ratio in the PLGA copolymer, with higher glycolide content typically resulting in faster degradation. For applications requiring specific degradation timeframes, the membrane's biodegradation profile can be optimized by selecting appropriate heat treatment parameters (temperature and duration) and polymer composition. Notably, heat treatment temperatures and durations can vary considerably depending on the particular polymer composition. This controlled biodegradability makes these membranes particularly suitable for temporary implantable devices, drug delivery systems, and tissue engineering scaffolds where a predetermined degradation timeline is desired.

### Example Glomerulus-On-a-Chip

The kidney glomerular barrier selectively filters blood as it passes through the afferent arteriole and into the Bowman's space. In their mature state, podocytes lose their proliferative capacity and develop specialized junctions between the cell body and the glomerular basement membrane (GBM), known as the focal adhesion complex, and junctions between foot processes called into the slit diaphragm. However, the functional basis for maintaining podocytes in their specialized state and their ability to sustain the filtration barrier is poorly understood. In this example, a platform was made involving co-culturing of glomerular podocytes and endothelial cells to assess GBM barrier function. Current in vitro glomerular filtration models have been challenging, particularly when evaluating albumin filtration or drug delivery due to the low porosity and non-uniform pore size of commercially available microporous membranes.

The example platform includes a GBM formed using a two-chamber organ chip embedding a biocompatible, high porosity, and uniform pore-sized PLGA (Poly Lactic-co-Glycolic Acid) membrane as described herein for co-culture of the podocytes and endothelial cells, maximizing cell-cell interaction to understand the formation of the slit diaphragm.

A glomerulus chip, consisting of two microfluidic chambers separated by a PLGA porous membrane and two access holes for transepithelial electrical resistance (TEER) measurement, was designed and fabricated. The chip had a design similar to FIG. 8 and FIG. 9, described above. Glomerular podocytes and endothelial cells were seeded on top and bottom chambers. The barrier function was evaluated by measuring TEER using the EVOM™ automated TEER instrument (World Precision Instruments, USA) and immunostaining to assess f-actin, vinculin, ZO-1, podocin, and nephrin expressions. Furthermore, DMSO (dimethyl sulfoxide) was used to evaluate the GBM barrier function by damaging the barrier.

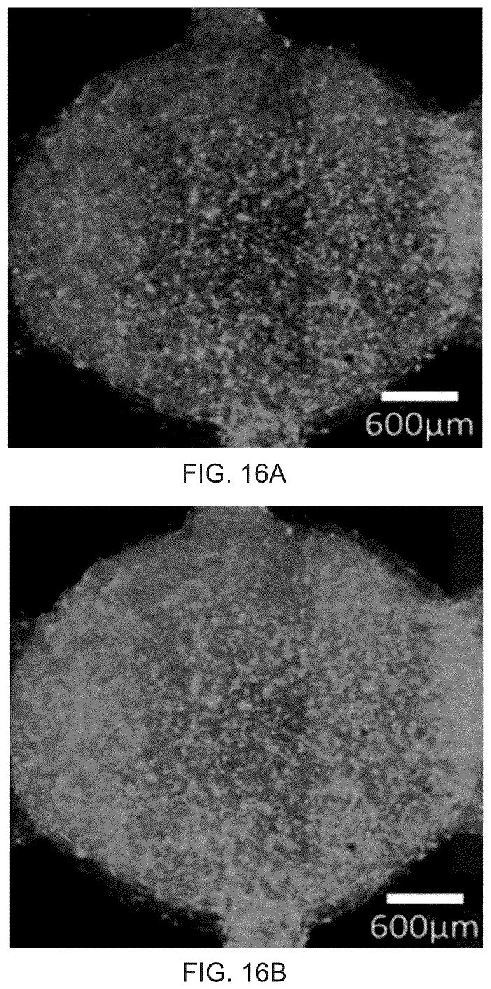

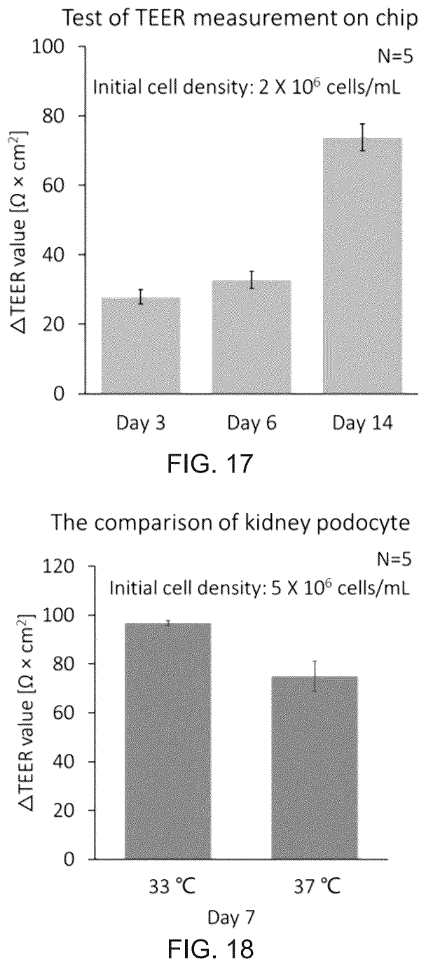

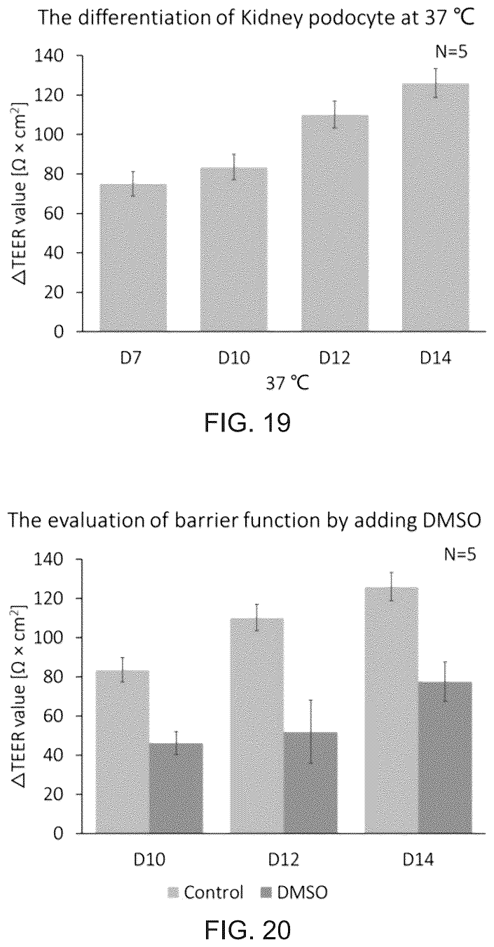

The highly porous PLGA membrane in the chip promoted cell-cell interactions and permeability, which are typically hindered by synthetic membranes. The chip provided a suitable environment for kidney podocytes cultured at 33° C. on the chip, as evidenced by an increasing TEER corresponding to cell proliferation. FIG. 16A shows a cell tracker image of the membrane after 6 days, and FIG. 16B shows a cell tracker image of the membrane after 14 days. FIG. 17 shows a graph of TEER measurements after 3 days, 6 days, and 14 days. Initially, differentiated cells at 37° C. exhibited collapsed tight junctions and decreased TEER compared to cells cultured at 33° C., as shown in the graph of FIG. 18. However, as differentiation progressed, podocytes recovered the TEER and showed enhanced focal adhesion, characterized by increased vinculin expression, enhanced cytoskeleton, and larger cell size, as shown in the graph of FIG. 19. The expression of nephrin and podocin, which are markers for maintaining a healthy slit diaphragm, also increased in differentiated podocytes. The glomerular filtration function with a slit diaphragm showed a correlation with the TEER. The differentiated kidney podocyte showed decreased ZO-1 expression compared to undifferentiated cells but significantly enhanced vinculin expression with thicker actin fibers. This complex networking structure, along with the formation of the slit diaphragm by increased nephrin and podocin, enabled selective protein filtration of the GBM. In contrast, TEER decreased after barrier damage with DMSO, as shown in graph of FIG. 20, suggesting that altered barrier function is closely linked to slit diaphragm properties, including tight junctions, focal adhesions, and gap junctions. Moreover, co-cultured glomerular endothelial cells can impact this barrier function by promoting slit diaphragm formation.

What is claimed is:

1. A porous membrane comprising:
a first plurality of near-field electrospun fibers that are substantially parallel one to another; and
a second plurality of near-field electrospun fibers that are substantially parallel one to another deposited over the first plurality of fibers, wherein the second plurality of fibers are transverse to the first plurality of fibers, such that the second plurality of fibers cross the first plurality of fibers to form pores between adjacent fibers of the first plurality of fibers and adjacent fibers of the second plurality of fibers.

2. The porous membrane of claim 1, wherein the first plurality of fibers are substantially straight and uniformly spaced one from another, and wherein the second plurality of fibers are substantially straight and uniformly spaced one from another.

3. The porous membrane of claim 1, wherein the second plurality of fibers are orthogonal to the first plurality of fibers.

4. The porous membrane of claim 1, wherein the second plurality of fibers are at least partially fused to the first plurality of fibers by a heat treatment.

5. The porous membrane of claim 1, wherein the first and second pluralities of fibers have an aspect ratio of fiber width to fiber thickness from about 1.5 to about 5.

6. The porous membrane of claim 1, wherein at least one of the first or second plurality of fibers have an average fiber width from about 1 μm to about 20 μm.

7. The porous membrane of claim 1, wherein at least one of:
the first plurality of fibers has an average fiber width that is different from an average fiber width of the second plurality of fibers; and
the first plurality of fibers is spaced at a first spacing distance, and wherein the second plurality of fibers is spaced at a second spacing distance that is different from the first spacing distance.

8. The porous membrane of claim 1, wherein the pores have an average pore size from about 1 μm to about 100 μm.

9. The porous membrane of claim 1, wherein at least 95% of the pores have a pore size within 10% of an average pore size.

10. The porous membrane of claim 1, wherein the membrane comprises multiple zones having a different average pore size in each of the zones.

11. The porous membrane of claim 1, wherein the membrane has a porosity from about 20% to about 80%.

12. The porous membrane of claim 1, further comprising a third plurality of near-field electrospun fibers that are substantially parallel one to another deposited over the second plurality of fibers, wherein the third plurality of fibers are transverse to first plurality of fibers and the second plurality of fibers.

13. The porous membrane of claim 1, wherein the membrane is a multi-layer membrane, wherein the first plurality of fibers and the second plurality of fibers form a first layer of the multi-layer membrane, and wherein the multi-layer membrane further comprises an additional layer formed by electrospinning separately from the first layer and at least partially fused to the first layer by heat treatment, wherein the additional layer comprises a third plurality of near-field electrospun fibers that are substantially parallel one to another and a fourth plurality of near-field electrospun fibers deposited over the third plurality of fibers transverse to the third plurality of fibers.

14. The porous membrane of claim 1, wherein at least one of the first plurality of fibers or the second plurality of fibers comprises polylactic acid (PLA), polyglycolic acid (PGA), poly(D,L-lactide-co-glycoside) (PLGA), polydimethylsiloxane (PDMS), polycarbonate (PC), polyvinylidene fluoride (PVDF), or a copolymer or combination thereof.

15. The porous membrane of claim 1, wherein the first plurality of fibers is made of a different polymer than the second plurality of fibers.

16. The porous membrane of claim 1, wherein the membrane has an anisotropic property that is different in a direction parallel to the first plurality of fibers than in a direction parallel to the second plurality of fibers.

17. The porous membrane of claim 1, wherein the membrane is biodegradable and has a controlled degradation rate that can be adjusted by heat treatment applied during manufacturing.

18. A drug delivery system comprising the porous membrane of claim 1, wherein the membrane is configured to release therapeutic agents at a controlled rate by controlling a degradation rate of the membrane.

19. A method of making a porous membrane, comprising:
electrospinning a first plurality of fibers using near-field electrospinning, wherein the first plurality of fibers are substantially parallel; and
electrospinning a second plurality of fibers over the first plurality of fibers using near-field electrospinning, wherein the second plurality of fibers are substantially parallel to each other, wherein the second plurality of fibers are transverse to the first plurality of fibers, such that the second plurality of fibers cross the first plurality of fibers to form pores between adjacent fibers of the first plurality of fibers and adjacent fibers of the second plurality of fibers.

20. The method of claim 19, wherein the electrospinning comprises ejecting a polymer fiber from a needle tip and collecting the fiber on a moving collector.

21. The method of claim 20, wherein the needle tip is positioned at a tip to collector distance from 0.05 mm to 10 mm during the electrospinning.

22. The method of claim 20, wherein the collector moves at a speed from 1 mm/s to 20 mm/s during the electrospinning.

23. The method of claim 20, wherein the electrospinning comprises applying a voltage between the needle tip and the collector, wherein the voltage is from 500 V to 1,000 V.

24. The method of claim 20, further comprising depositing a sacrificial layer on the collector before the electrospinning, wherein the sacrificial layer comprises polyethylene oxide (PEO), polyvinyl alcohol (PVA), or a combination thereof.

25. The method of claim 19, further comprising heat treating the porous membrane at a temperature from about 50° C. to about 100° C.

26. The method of claim 25, wherein the porous membrane is heated at the temperature for a time from 5 minutes to 60 minutes.

27. A tissue chip, comprising:
a substrate;
a porous membrane supported by the substrate, wherein the porous membrane comprises:
a first plurality of near-field electrospun fibers that are substantially parallel one to another, and
a second plurality of near-field electrospun fibers that are substantially parallel one to another deposited over the first plurality of fibers, wherein the second plurality of fibers are transverse to the first plurality of fibers, such that the second plurality of fibers cross the first plurality of fibers to form pores between adjacent fibers of the first plurality of fibers and adjacent fibers of the second plurality of fibers; and
cultured cells supported by the porous membrane.
